(19) United States
(12) Patent Application Publication
(10) Pub. No.: US 2022/0152232 A1
(43) Pub. Date: May 19, 2022

(54) Germicidal Light Dosage Dispensing System

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/099,271

(22) Filed: Nov. 16, 2020

(51) Int. Cl. A61L 2/08; A61L 2/26; A61L 2/10

(52) U.S. Cl. CPC A61L 2/084; A61L 2/26; A61L 2202/14; A61L 2202/11; A61L 2/10

(57) ABSTRACT

A germicidal light dosage dispending system includes at least one light source, one driver for each at least one light source, and a germicidal light dosage dispensing mechanism. The at least one light source is a germicidal light source emitting a light with a spectral power distribution (SPD) >90% in the wavelength range 190˜420 nm. The driver converts an external power to an internal power for activating the at least one light source and is controllable by the germicidal light dosage dispensing mechanism. The germicidal light dosage dispensing mechanism limits the total dispensed dosage emitted by the at least one light source over an 8-hour period to be less than the ACGIH defined UV Threshold Limit Value (TLV) dosage. The germicidal light dosage dispensing mechanism also controls the total dispensed dosage.

### BACKGROUND

### Technical Field

The present disclosure pertains to germicidal lighting devices and, more specially, proposes a germicidal light dosage dispensing system.

### Description of Related Art

Germicidal lighting refers to the use of a light source emitting primarily ultraviolet (UV) wavelength in a range of 190 nm˜420 nm (with a peak performance at 254 nm) for disinfecting against bacteria and viruses in the air or on a surface. Germicidal lighting applications are not new. There is, however, a renewed interest of the germicidal lighting technologies and applications due to the COVID-19 pandemic. It is shown that a UVC dosage of 5 mJ/cm2 can disinfect against the SARS-CoV-II virus (the COVID-19 virus) with a 99.99% kill rate. This gives rise to the proliferation of germicidal lighting devices on the market. However, most of these devices failed to address two critical, and somewhat contradictory, requirements. The first requirement is how to ensure a germicidal lighting device could provide a sufficient germicidal light dosage for effectively disinfecting a pathogen. The second requirement is how to ensure a germicidal lighting device would not over-expose a user to UV wavelength, given that a UV light can cause skin and eye damages with an over-dosage.

Recent studies have demonstrated that a far-UVC light source emitting a light with a wavelength range 200˜230 nm has the effect of killing bacteria and viruses, yet without the side effect of causing skin and eye damages to a user. One such study can be found at https://www.cuimc.columbia.edu/news/far-uvc-light-safely-kills-airborne-coronaviruses.

This leads to the possibility of using a far-UVC light source with a 200 nm˜230 nm wavelength range in germicidal lighting equipment. However, it is still possible to over-dose a user with far-UVC. American Conference of Governmental Industrial Hygienists (ACGIH) has published a UV Safety Guidelines as shown in FIG. 1 (ACGIH ISBN: 0-9367-12-99-6). It shows the UV Threshold Limit Values (TLVs), which is the maximum allowable dosage (in mJ/cm²) for each wavelength over an 8-hour period. It can be seen from FIG. 1, the TLV for 222 nm wavelength is set to 22 mJ/cm². Therefore, even with a 222 nm germicidal light source, it is not recommended to administrate more 22 mJ/cm² for an 8-hour workday.

The present disclosure proposes a germicidal light dosage dispensing system that aims at striking a balance between the two requirements mentioned above and achieving an effective disinfection through germicidal lighting but without the side effects of skin or eye damages. This system is applicable to any wavelength and for multiple germicidal light sources simultaneously.

### SUMMARY

The radiant power is the radiant energy emitted by a germicidal light source and is measured in milli-Watts or mW. The irradiance is defined as the radiant energy per unit area, measured in mW/cm². The Germicidal Light Dosage, or simply Dosage, can be defined as:

Dosage (mJ/cm²)=Irradiance (mW/cm²)×Time (second)

From this definition, it can be seen that the germicidal light dosage depends on two factors: the irradiance and the time (of exposure under a given irradiance). To dispense a certain germicidal light dosage, e.g., 5 mJ/cm², one can use a high radiant power germicidal light source (resulting in a higher irradiance) with a short exposure time or use a low radiant power germicidal light source (resulting in a lower irradiance) with a longer exposure time. The essence of the present disclosure lies in its novelty of manipulating these two factors, the irradiance and the exposure time, without exceeding the ACGHI UV Safety Guidelines.

In one aspect, the germicidal light dosage dispending system comprises at least one light source, one driver for each of the at least one light source, and a germicidal light dosage dispensing mechanism. If the system has two light sources, then there will be two drivers, one for each of the two light sources. The driver converts an external power to an internal power for activating the at least one light source and is controllable by the germicidal light dosage dispensing mechanism. The at least one light source is a germicidal light source and is configured to emit a light with a spectral power distribution (SPD) >90% in the wavelength range 190˜420 nm. The germicidal light dosage dispensing mechanism is configured to limit the Total Dispensed Dosage, TDD, (in mJ/cm²) emitted by the at least one light source over a predefined period (e.g., an 8-hour period) not to exceed the ACGIH defined UV Threshold Limit Value (TLV) dosage. The ACGIH defined UV TLV dosage is shown in FIG. 1. For example, if the at least one light source emits primarily a 222 nm wavelength, then the germicidal light dosage dispensing mechanism is configured to limit the Total Dispensed Dosage DDISP to 22 mJ/cm² or less. Similarly, if the at least one light source emits primarily a 254 nm wavelength, then the germicidal light dosage dispensing mechanism is configured to limit the TDD to 6 mJ/cm² or less. Moreover, the germicidal light dosage dispensing mechanism controls the TDD (mJ/cm²) by either by limiting the operating time of the at least one light source during the predefined period, or by reducing the radiant power (mW) emitted by the at least one light source, or a combination thereof.

The light source may emit a light in the UVC wavelength (190-280 nm), the UVB wavelength (280-315 nm), the UVA wavelength (315-400 nm), the near-UV wavelength (400-420 nm), or a combination thereof. In some embodiments, the at least one light source is a far-UVC light source and is configured to emit a light with a spectral power distribution (SPD) >90% in the wavelength range 190˜230 nm. In some other embodiments, the at least one light source is a regular UVC light source and is configured to emit a light with a spectral power distribution (SPD) >90% in the wavelength range 230˜280 nm. In some other embodiments, the at least one light source is a regular UVA light source and is configured to emit a light with a spectral power distribution (SPD) >90% in the wavelength range 315˜400 nm. In some other embodiments, the at least one light source is a near-UV light source and is configured to emit a light with a spectral power distribution (SPD) >90% in the wavelength range 390˜420 nm.

The light emitting diode (LED) is a good narrow-band light source. It may be used for making far-UVC LED, UVC LED, UVA LED, or near-UV LED light source. Therefore, in some embodiments, the at least one light source comprises one or more light emitting diodes (LEDs). Other light sources, such as low-pressure mercury lamp, xenon arc lamp, or excimer lamp may also be used for the at least one light source.

In some embodiments, the germicidal light dosage dispensing mechanism is configurable according to the distance between the at least one light source to a surface to be disinfected. For a germicidal light source to deliver a certain germicidal light dosage, e.g., 5 mJ/cm², to a desktop 2.5-ft above the floor, it would take that light source a lesser time to dispense such dosage when mounted at a 9-ft ceiling, as compared to the time needed when the light source is mounted at a 12-ft ceiling. This is because the spectral power drops with the increase of distance.

A light source with an adjustable radiant power that may be tuned according to the distance between the light source and a surface to be disinfected could then be used to deliver a target germicidal light dosage regardless the distance. Therefore, in some embodiments, the germicidal light dosage dispensing mechanism is configurable by adjusting the radiant power (mW) emitted by the at least one light source according to the distance of the at least one light source to a surface to be disinfected. More specifically, in some embodiments, the germicidal light dosage dispensing mechanism has at least two radiant power (mW) settings for the at least one light source where a lower radiant power setting is used when the light source is closer to a surface to be disinfected (e.g., 3-ft), and a higher radiant power setting is used when the light source is farther to a surface to be disinfected (e.g., 6-ft).

In some embodiments, the germicidal light dosage dispensing mechanism is configurable by adjusting the operating time of the at least one light source according to the distance of the at least one light source to a surface to be disinfected. More specifically, in some embodiments, the germicidal light dosage dispensing mechanism has at least two operating duration settings for the at least one light source such that the light source operates for a shorter duration when the light source is closer to a surface to be disinfected, and the light source operates for a longer duration when the light source is farther to a surface to be disinfected.

In some embodiments, the germicidal light dosage dispensing mechanism is configured to operate the at least one light source intermittently. For example, in order to dispense 22 mJ/cm² over 8 hours, the germicidal light dosage dispensing mechanism may dispense 22 mJ/cm²/8 hours=2.75 mJ/cm² per hour by operating the at least one light source for 5 minutes on top of the hour for 8 hours.

In some embodiments, the germicidal light dosage dispensing mechanism is configured to operate the at least one light source continuously. For example, in order to dispense 22 mJ/cm² over 8 hours continuously, the germicidal light dosage dispensing mechanism may dispense 22 mJ/cm²/(8 hours×60 minutes×60 seconds)=0.7638 pJ/cm² per second by operating the at least one light source continuously at this dosage level.

In some embodiments, the germicidal light dosage dispensing mechanism is configured to operate the at least one light source in a full sanitation mode by operating the at least one light source at its maximum radiant power for delivering a UV dosage greater than the ACGIH defined UV TLV dosage. In other words, the germicidal light dosage dispensing mechanism may dispense the Maximum Dosage DMAX ( mJ/cm²) in a short burst for quick disinfection of the environment. For example, the ACGIH defined UV TLV dosage is 22 mJ/cm² at 222 nm over an 8-hour period. During the full sanitation mode, the germicidal light dosage dispensing mechanism may dispense 3×22 mJ/cm²=66 mJ/cm² in one hour.

In some embodiments, an operation schedule of the at least one light source comprises one full sanitation mode and either one intermittent mode or one continuous mode over a 24-hour period. For example, an operation schedule may include one 8-hour continuous mode for working hours (e.g., 9 am to 5 pm) and 1-hour full sanitation mode for off hours (e.g. 5 pm to 6 pm).

In some embodiments, the present disclosure further comprises a motion sensor. Upon a motion detection the motion sensor shuts off the at least one light source operating in the full sanitation mode. This is to prevent any occupant from being dosed with the germicidal light. After detecting no more motions, the germicidal light dosage dispensing mechanism may resume the operation of the full sanitation mode.

In some embodiments, the germicidal light dosage dispensing mechanism is configured to store an operation schedule of the at least one light source.

In some embodiments, the germicidal light dosage dispensing mechanism may comprise a controller module connecting directly to the driver of the at least one light source. Alternatively, in some embodiments, the germicidal light dosage dispensing mechanism comprises a remote lighting control system, a controller module connecting directly to the driver of the at least one light source, and a communication mechanism for communication the operation information between the remote lighting control system and the controller module. The remote lighting control system may be an app on a smartphone, and it can communicate with the at least one light source wirelessly via WIFI or Bluetooth. A user may use the app to set the operating schedule for one or more germicidal light sources. The remote lighting control system may be a centralized lighting control system capable of setting the operation schedule of the at least one light source through a wired or wireless communication means. The operation schedule may differ for working hours (e.g., 8-hour continuously mode or 8-hour intermittent mode) and off hours (e.g., 1-hour full sanitation mode). Moreover, the remote lighting control system may control multiple germicidal light sources where each may have a different operation schedule and may dispense a different amount of germicidal light dosage, and each germicidal light source may emit a different radiant power and be mounted at a different ceiling height.

In some embodiments, the germicidal light dosage dispensing mechanism is configured to store the Illuminating Engineering Society (IES) data of the at least one light source and to use the IES data for calculating the maximum dosage of the at least one light source without exceeding the predefined UV TLV dosage. Each germicidal light source has a different radian power, a different spectral power distribution, and a different irradiance, thus resulting in a different germicidal light dosage at a different distance over a different duration. By using the IES data, the germicidal light dosage dispensing mechanism can calculate accurately the maximum germicidal light dosage that may be administrated into a space without exceeding the ACGIH defined UV TLV dosage. The IES data may be stored in a smartphone app or in a centralized lighting control system for administrating multiple germicidal light sources.

In some embodiments, the germicidal light dosage dispensing mechanism is configured to store the distance of the at least one light source to a surface to be disinfected. The distance information is used for calculating the maximum dosage of the at least one light source without exceeding the ACGIH defined UV TLV dosage. For a germicidal light source with a fixed radiant power, it takes different durations to deliver the same germicidal light dosage at different distances. By factoring in the distance (e.g., mounting height) of the at least one light source to a surface (e.g., a desktop) to be disinfected, the germicidal light dosage dispensing mechanism may maximize the dosage of the germicidal light dosage accurately without exceeding the ACGIH defined UV TLV dosage. The distance information may be stored in a smartphone app or in a centralized lighting control system for administrating multiple germicidal light sources.

In some embodiments, the germicidal light dosage dispensing mechanism is configured to store the ACGIH defined UV TLV dosage. The ACGIH defined UV TLV dosage may be stored in a smartphone app or in a centralized lighting control system for administrating multiple germicidal light sources. Alternatively, the ACGIH defined UV TLV dosage may be embedded in a controller module directly connecting to the driver of the at least one light source.

When the ACGIH defined UV TLV data is stored in a smartphone app or in a centralized lighting control system for administrating multiple germicidal light sources, there may be situations that call for a different UV TLV dosage. For example, for high traffic areas, such as airports, subways, commuter buses, or elevators, occupants will not stay in the same space for long. However, many occupants would come in and out through the space, thus these high traffic areas may be a potential hotspot for contracting infectious diseases. For these areas, it may be reasonable to double the ACGIH defined UV TLV dosage. This is because an occupant is unlikely to stay in the same high traffic area (e.g. a subway or a commuter bus or an elevator) more than 4 hours, and his/her germicidal light exposure would still within ACGIH UV Safety Guidelines over an 8-hour period. Conversely, there may be situations calling for reducing ACGIH defined UV TLV dosage, e.g., for patients hyper-sensitive to UV light. Therefore, in some embodiments, the germicidal light dosage dispensing mechanism is configured to increase or decrease ACGIH defined UV TLV dosage. More specifically, in some embodiments, the germicidal light dosage dispensing mechanism is configured to at least double the ACGIH defined UV TLV dosage. The ACGHI defined UV TLV dosage settings can be easily reconfigured when they are stored in a smartphone app or in a centralized lighting control system.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

FIG. 2 schematically depicts a diagram of a continuous germicidal lighting device of the present disclosure.

FIG. 3 schematically depicts a diagram of another germicidal lighting device of the present disclosure with a motion sensor and three different configurations based on the mounting height.

FIG. 4 shows an operation schedule of a second germicidal lighting device of the present disclosure with three different configurations based on the mounting height.

FIG. 5 shows two germicidal lighting devices of the present disclosure mounted at different ceiling heights controllable by one smartphone app.

FIG. 6. schematically depicts a diagram of a continuous germicidal lighting device of the present disclosure controllable by a smartphone app.

FIG. 7 schematically depicts a user interface of a smartphone app for controlling a continuous germicidal lighting device of the present disclosure.

FIG. 8. schematically depicts a diagram of another germicidal lighting device of the present disclosure with a motion sensor and controllable by a smartphone app.

FIG. 9 schematically depicts a user interface of a smartphone app for controlling a germicidal lighting device of the present disclosure with a motion sensor.

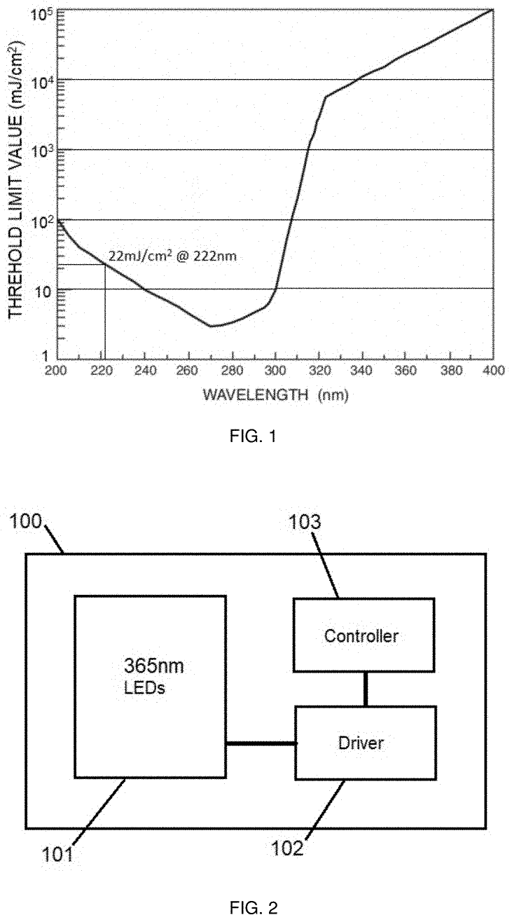

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting devices having different form factors.

The present disclosure germicidal light dosage dispending system includes at least one light source, one driver for each at least one light source, and a germicidal light dosage dispensing mechanism. The at least one light source is a germicidal light source emitting a light with a spectral power distribution (SPD) >90% in the wavelength range 190˜420 nm. The driver converts an external power to an internal power for activating the at least one light source and is controllable by the germicidal light dosage dispensing mechanism. The germicidal light dosage dispensing mechanism is configured to limit the total dispensed dosage emitted by the at least one light source over a predefined period (e.g., an 8-hour period) to be less than the ACGIH defined UV Threshold Limit Value (TLV) dosage. The germicidal light dosage dispensing mechanism controls the total dispensed dosage by either by limiting the operating time of the at least one light source during the predefined period, or by reducing the radiant power emitted by the at least one light source, or a combination thereof.

### Example Implementations

FIG. 2 is an embodiment of the lighting device of the present disclosure in a form of a standalone germicidal lighting device 100. The device 100 includes one LED light source 101, one driver 102, and a controller 103. The driver 102 converts an external power to an internal power for activating the LED light source 101. The LED light source comprises multiple 365 nm LEDs. Though not shown, the controller 103 has a fixed, built-in schedule that operates the LEDs light source 101 at 10% radian power from 07:00 to 19:00, and at 100% radian power from 19:00 to 07:00. The maximum germicidal light dosage of the LED light source 101 is chosen to be below the ACGHI defined UV TLV at 365 nm (˜2.5×10⁴ mJ/cm²) at any distance.

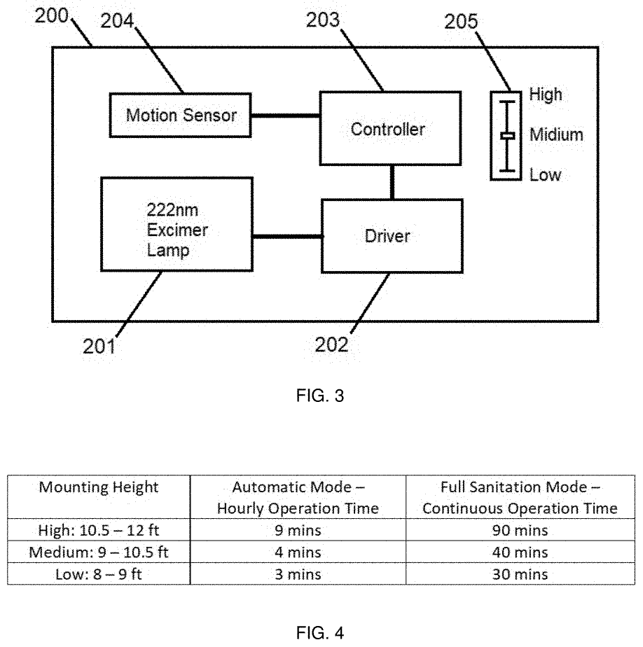

FIG. 3 is another embodiment of the lighting device of the present disclosure in a form of a standalone germicidal lighting device 200. The device includes one excimer light source 201, one driver 202, a controller 203, a motion sensor 204, and a slide switch 205 to be set according to the mounting height of the device. The driver 202 converts an external power to an internal power for activating the excimer light source 201. The excimer light source emits a light primarily at 222 nm wavelength. This device 200 is designed to operate in two modes: the automatic mode and the full sanitation mode. When the device is turned on, it enters the automatic mode. If a user turns the device On-Off-On in sequence within 2 seconds, the device enters the full sanitation mode. When in the automatic mode, the controller 203 operates the excimer light source 201 intermittently to dispense 22 mJ/cm²/8 hours=2.75 mJ/cm² every hour in the beginning of the hour. The operation time of the excimer light source 201 to dispense the target dosage 2.75 mJ/cm² to a desktop surface (2.5-ft from the floor) varies according to the mounting height of the device 200. The slide switch 205 supports three different mounting height settings. The table in FIG. 4 shows an example operation schedule of the device 200 according to the mouthing height. When the slide switch 205 is set to High, corresponding to a 10.5-12 ft ceiling, the controller 203 would operate the excimer light source 201 for 9 minutes in order to dispense 2.75 mJ/cm² to a desktop surface (2.5-ft from the floor). When the slide switch 205 is set to Low, corresponding to a 8-9 ft ceiling, the controller 203 would operate the excimer light source 201 for 3 minutes in order to dispense 2.75 mJ/cm² to a desktop surface (2.5-ft from the floor). When in the full sanitation mode, the controller 203 would operate the excimer light source 201 continuously based on the duration listed in the table in FIG. 4. When in the full sanitation mode, the motion sensor 204, upon detecting a motion, would inform the controller 203 to suspend the operation of the full sanitation mode. When there is no further motion detected, the controller 203 would resume the operation of the full sanitation mode. After the completion of the execution of the full sanitation mode, the controller would return the device 200 to the automatic mode.

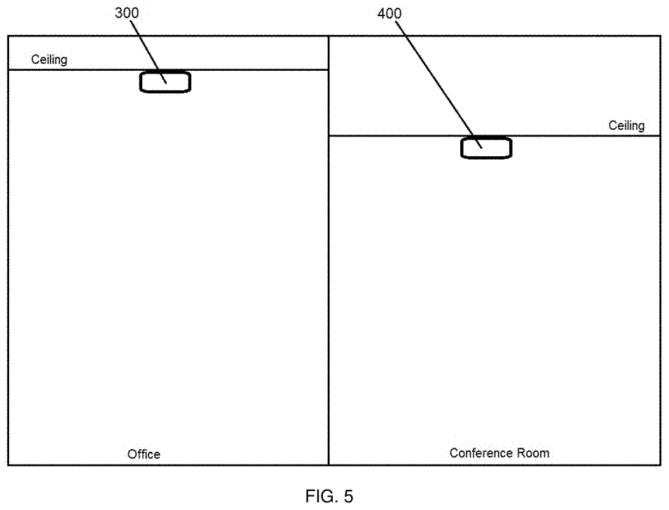

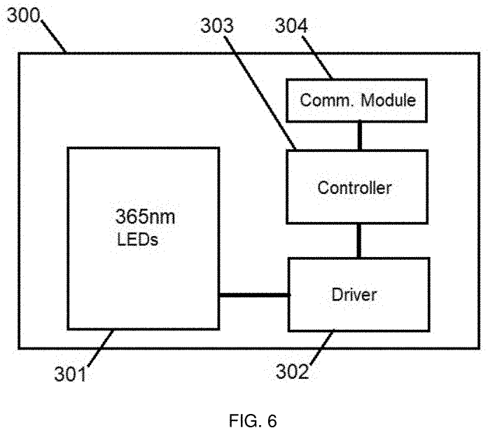

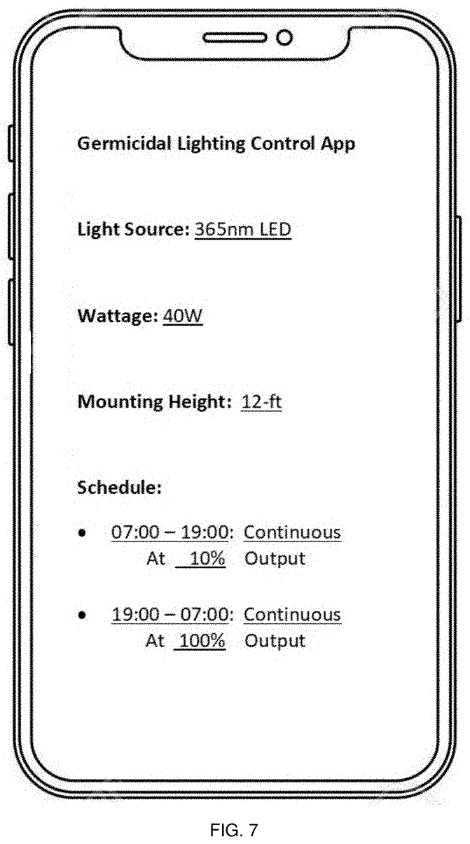

FIG. 5 shows a scenario where a facility has two rooms with different ceiling heights. The office has a taller ceiling and uses a germicidal lighting device 300. The conference room has a shorter ceiling and uses a germicidal lighting device 400. FIG. 6 shows the device 300 comprising a 365 nm LED light source 301, a driver 302, a controller 303, and a communication module 304. The communication module 304 communicates with the smartphone app wirelessly. The device 300 is controllable remotely via a smartphone app through the communication module 304. The user interface of the smartphone app for controlling the device 300 is shown in FIG. 7. The smartphone app can select the light source. In this case, a 365 nm LED light source is selected. The wattage of the light source 301 is specified to be 40 W and the mounting height is set to be 12-ft. The app allows a user to set the operation schedule and the output level of the 365 nm LED light source 301, e.g., 10% output level from 07:00-19:00 and 100% output level from 19:00-07:00.

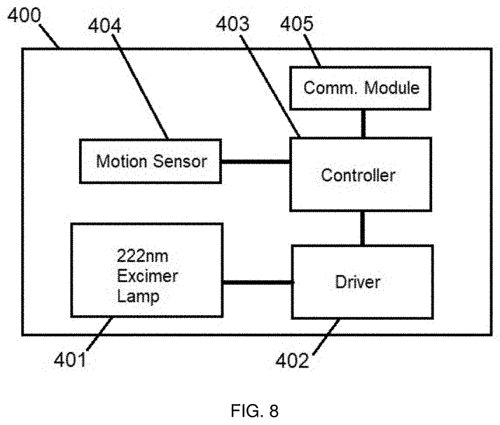

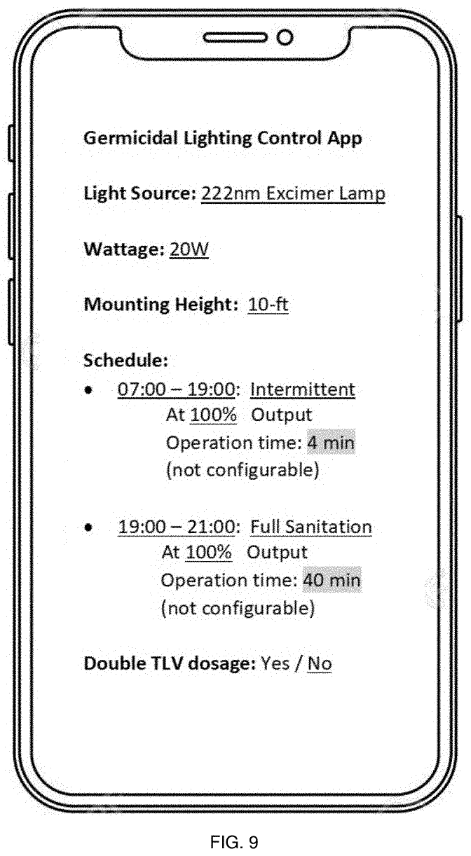

FIG. 8 shows the device 400 comprising a 222 nm excimer lamp 401, a driver 402, a controller 403, a motion sensor 404, and a communication module 405. The device 400 is controllable remotely via the same smartphone app controlling the device 300. The communication module 405 communicates with the smartphone app wirelessly. The user interface of the smartphone app for controlling the device 400 is shown in FIG. 9. The smartphone app can select the light source. In this case, a 222 nm excimer lamp is selected. The wattage of the light source 401 is specified to be 20 W and the mounting height is set to be 10-ft. The app allows a user to set the operation schedule, e.g., 0700:19:00 in the automatic mode. Though not shown in FIG. 9, the ACGIH defined UV TLV dosage is stored in the smartphone app. Also stored in the smartphone app is the IES data of the 222 nm excimer lamp, though not shown. Using the mounting height of the device 400 and the IES data of the light source 401, the smartphone app calculates the hourly germicidal light dosage the light source 401 could dispense intermittently every hour (2.75 mJ/cm²) to a desktop (2.5-ft from the floor) without exceeding the ACGIH defined UV TLV dosage (22 mJ/cm²) over an 8-hour period. The operation time “4-min” shown in FIG. 9 is calculated by the smartphone app and is not configurable by a user. A user can also set the schedule for the full sanitation mode. When in the full sanitation mode, the smartphone app calculates the germicidal light dosage according to the IES data of the light source 401, the mounting height, and the ACGIH defined UV TLV dosage pertaining to the wavelength of the light source 401, and sets the operation duration of the full sanitation mode accordingly, e.g., “40-min” in this case. This is not configurable by a user. When in the full sanitation mode, the motion sensor 404, upon detecting a motion, would inform the controller 403 to suspend the operation of the full sanitation mode. When there is no further motion detected, the controller 403 would resume the operation of the full sanitation mode. After the completion of the execution of the full sanitation mode, the controller would return the device 400 to the automatic mode. The smartphone app would also allow a user to double the TLV dosage when necessary, as shown on the bottom of FIG. 9. This is to accommodate the situation when there are a lot of people going in and out the conference room in a short period.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A germicidal light dosage dispending system, comprising a light source;
a driver for the light source; and
a germicidal light dosage dispensing mechanism configured to control the driver, wherein:
the driver converts an external power to an internal power to activate the light source,
the light source comprises a germicidal light source and is configured to emit a light with a spectral power distribution (SPD) >90% in a wavelength range of 190˜420 nm,
the germicidal light dosage dispensing mechanism is configured to limit a total dispensed dosage (TDD) emitted by the light source over a predefined period not to exceed an ultraviolet (UV) threshold limit value (TLV) dosage defined by American Conference of Governmental Industrial Hygienists (ACGIH), and
the germicidal light dosage dispensing mechanism controls the TDD by either or both of:
limiting an operating time of the light source during the predefined period, and
reducing a radiant power emitted by the light source.

2. The germicidal light dosage dispending system of claim 1, wherein the light source is configured to emit a light with a SPD>90% in a wavelength range of 190˜230 nm.

3. The germicidal light dosage dispending system of claim 1, wherein the light source is configured to emit a light with a SPD>90% in a wavelength range of 230˜280 nm.

4. The germicidal light dosage dispending system of claim 1, wherein the light source is configured to emit a light with a SPD>90% in a wavelength range of 315˜400 nm.

5. The germicidal light dosage dispending system of claim 1, wherein the light source is configured to emit a light with a SPD>90% in a wavelength range of 390˜420 nm.

6. The germicidal light dosage dispending system of claim 1, wherein the light source comprises one or more light emitting diodes (LEDs).

7. The germicidal light dosage dispending system of claim 1, wherein the germicidal light dosage dispensing mechanism is configurable according to a distance between the light source and a surface to be disinfected.

8. The germicidal light dosage dispending system of claim 7, wherein the germicidal light dosage dispensing mechanism is configurable by adjusting the radiant power emitted by the light source according to the distance.

9. The germicidal light dosage dispending system of claim 8, wherein the germicidal light dosage dispensing mechanism has at least two radiant power settings for the light source, wherein a lower radiant power setting of the at least two radiant power settings is used when the light source is closer to the surface to be disinfected, and wherein a higher radiant power setting of the at least two radiant power settings is used when the light source is farther from the surface to be disinfected.

10. The germicidal light dosage dispending system of claim 7, wherein the germicidal light dosage dispensing mechanism is configurable by adjusting the operating time of the light source according to the distance.

11. The germicidal light dosage dispending system of claim 10, wherein the germicidal light dosage dispensing mechanism has at least two operating duration settings for the at least one light source, wherein the light source operates for a shorter duration when the light source is closer to the surface to be disinfected, and wherein the light source operates for a longer duration when the light source is farther from the surface to be disinfected.

12. The germicidal light dosage dispending system of claim 1, wherein the germicidal light dosage dispensing mechanism is configured to operate the light source intermittently.

13. The germicidal light dosage dispending system of claim 1, wherein the germicidal light dosage dispensing mechanism is configured to operate the light source continuously.

14. The germicidal light dosage dispending system of claim 1, wherein the germicidal light dosage dispensing mechanism is configured to operate the light source in a full sanitation mode by operating the light source at its maximum radiant power to deliver a UV dosage greater than the UV TLV dosage defined by the ACGIH.

15. The germicidal light dosage dispending system of claim 14, wherein an operation schedule of the light source comprises one full sanitation mode and either one intermittent mode or one continuous mode over a 24-hour period.

16. The germicidal light dosage dispending system of claim 15, further comprising:
a motion sensor wherein, upon motion detection of a motion, the motion sensor is configured to shut off the light source operating in the full sanitation mode.

17. The germicidal light dosage dispending system of claim 1, wherein the germicidal light dosage dispensing mechanism is configured to store an operation schedule of the light source.

18. The germicidal light dosage dispending system of claim 1, wherein the germicidal light dosage dispensing mechanism comprises a controller module connecting directly to the driver of the light source.

19. The germicidal light dosage dispending system of claim 1, wherein the germicidal light dosage dispensing mechanism comprises a remote lighting control system, a controller module connecting directly to the driver of the light source, and a communication mechanism configured to communicate operation information between the remote lighting control system and the controller module.

20. The germicidal light dosage dispending system of claim 1, wherein the germicidal light dosage dispensing mechanism is configured to store Illuminating Engineering Society (IES) data of the light source and to use the IES data in calculating a maximum dosage of the light source without exceeding the UV TLV dosage defined by the ACGIH.

21. The germicidal light dosage dispending system of claim 1, wherein the germicidal light dosage dispensing mechanism is configured to store a distance between the light source and a surface to be disinfected.

22. The germicidal light dosage dispending system of claim 1, wherein the germicidal light dosage dispensing mechanism is configured to store the UV TLV dosage defined by the ACGIH.

23. The germicidal light dosage dispending system of claim 1, wherein the germicidal light dosage dispensing mechanism is configured to increase or decrease the UV TLV dosage defined by the ACGIH.

24. The germicidal light dosage dispending system of claim 23, the germicidal light dosage dispensing mechanism is configured to at least double the UV TLV dosage defined by the ACGIH.
